(19) United States
(12) Patent Application Publication
(10) Pub. No.: US 2008/0009844 A1
(43) Pub. Date: Jan. 10, 2008

(54) Device for Laser Surgery

(76) Inventors: Ingeborg Rolle, Mammendorf (DE); Johannes Bernhard Koeth, Gerbrunn (DE)

Correspondence Address: PLUMSEA LAW GROUP, LLC, 10411 MOTOR CITY DRIVE, SUITE 320, BETHESDA, MD, 20817, US

(21) Appl. No.: 11/426,415

(22) Filed: Jun. 26, 2006

(51) Int. Cl. A61B 18/18

(52) U.S. Cl. 606/16; 606/3

(57) ABSTRACT

The invention refers to a device for laser surgery comprising a laser and means for coupling laser light of the laser into an optical fiber, wherein the laser is a solid-state layer, which may emit laser light which at maximum intensity has a wavelength in the ragne of 1100 and 1400 nm, wherein the laser has an output power of at least 25 W.

The present invention refers to a device for laser surgery, comprising a laser and means for coupling laser light of the laser into an optical fiber.

Devices of this type for laser surgery are for instance known from DE 91 162 16 U. DE 44 08 746 C2 also shows a laser catheter for bypass surgery. In this case Nd: YAG lasers are for instance mentioned. These lasers have a laser line at 1064 nm.

It is the object of the present invention to improve a device for laser surgery. This object is solved by a device according to claim 1.

For laser surgery of tissues in general, but particularly for the surgery of pulmonary tissue, it emerged that a wavelength between 1100 nm and 1400 nm is advantageous. In this wavelength range an absorption of the laser irradiation in aqueous tissue, i.e. also pulmonary tissue, sets in, which allows to efficiently separate or cut the tissue while at the same time generating an expanded coagulation on severely blood-supplied tissue. In pulmonary tissue, a further very important effect is at the same time achieved, namely the welding of air fistula. Although laser systems are available in this wavelength range, however, these systems are usually very unhandy, since for instance gas lasers usually require a complex water cooling so that the device of this kind is very heavy or the outputs are too low.

For laser surgery in the above-mentioned wavelength range a laser with an output of at least 25 W is desirable.

Due to recent material developments, it is possible to provide solid-state lasers such as semi-conductor lasers and/or fiber lasers with such outputs, as they are required for laser surgery, particularly for pulmonary surgery and to generate a wavelength in the range of 1100 to 1400 nm.

A wavelength range above 1150, 1175, 1200, 1250, 1275, 1300, 1303 or 1310 nm is especially preferred. In this wavelength range the absorption of water does not reveal such a great dependency on the wavelength, so that certain wavelength fluctuations in this area do not automatically lead to a different surgical behavior of the laser irradiation.

On the other hand, the laser irradiation should also be below a wavelength of 1350, 1330, 1325, 1300, 1275, 1250, 1225 or 1200 nm, since otherwise the plateau range is left. On this plateau range a relatively favorable absorption and an advantageous ratio of absorption to dispersion in tissues with a relatively high water content and thus also in pulmonary tissue sets in.

Furthermore, lasers are advantageous with a laser light that has a certain spectral full width at half maximum. Here, the spectral range of the spectrum is determined at half of the maximum intensity. The full width at half maximum is for instance at least 2, 3, 4, 5, 7, 10, 12 or 15 nm. The upper limit for the full width at half maximum may for instance be 2, 23, 4, 5, 7, 10, 12, 15, 20, 25, 30 or 40 nm. By covering a larger spectral range, the device becomes insensitive to small wavelength fluctuations, so that a constant surgical behavior of the laser light during surgery is ensured. On the other hand, the spectrum shall not be too broad so that spectral portions with an absorption in the tissue that is too high do not exist.

The output power of the laser is preferably greater than 25 W. In this case a power of e.g. at least 80 W is preferred.

The laser may be both a continuous-wave laser or a pulsed laser.

Preferably, a laser that comprises a plurality of laser elements is also advantageous. These may be at least or exactly 2, 5, 10, 15, 19, 20, 25, 30, 35, 38, 40, 50, 60, 70, 80, 90, 100, 150 laser elements. The individual laser elements can be operated with a lower power than with the desired overall output power, which increases life of these laser elements or which enables greater output powers.

Furthermore, means are advantageously provided, by means of which the laser beams of these individual laser elements can be coupled into one common optical fiber. It is also conceivable to provide one single optical fiber for each laser element. These individual fibers can then be bundled and the light of same can be joined with an optical system only at the end of the optical fiber. Such means for coupling-in laser light may comprise one or several lenses and/or mirrors, wherein cylindrical lenses/lens arrays and/or mirrors can also be provided.

The individual laser elements and/or lasers may be or comprise semiconductor lasers and/or fiber lasers. Individual fiber lasers may for instance be arranged in a bundled manner to give the light into a common output optical fiber. One or several fiber couplers may also be provided by means of which the light of two or more fibers is coupled into one single fiber.

The output power required can also be achieved by semiconductor lasers in the desired wavelength range. For this purpose, the light of a plurality of laser elements is coupled into an optical fiber via a beam optical system.

The semiconductor lasers may be pumped either electrically or optically. An optical pumping may for instance be implemented by other semiconductor lasers with a wavelength shorter than 1100 nm. The fiber lasers are usually pumped optically, such as by semiconductor laser diodes, as they are described in this document. Usually, the pump lasers will have a wavelength of below 1100 nm, however at least below the wavelength of the fiber laser.

Advantageously, the laser is a quantum dot laser or a quantum film laser. By such laser elements it is possible by using a reasonable amount of laser elements to provide the required output power in the desired wavelength range. Such quantum dot lasers may be based on the GaAs-AlGaAs material system. The quantum dots of the quantum dot laser may be provided in quantum films (also referred to as troughs) so that the confinement of the charge carriers in the area of the quantum dots is improved by the confinement of the charge carriers in the quantum film.

Quantum dots may, however, also be provided without quantum films or outside of possible quantum films.

Quantum dots may comprise or consist of GaInAs, GaInAsN and/or GaInSb. The quantum dots have a bandgap, which is smaller than the one of the surrounding material. Thereby the charge carriers are brought to energy levels, which are amongst others predetermined by the size of the quantum dot so that laser wavelengths also become possible in this case that do not correspond to the bandgap of the quantum dot material.

The waveguide of the semiconductor laser determines the light guidance within the semiconductor material. A relatively broad waveguide is preferred for the laser for the device for laser surgery, since the light extends over a large range in the semiconductor material and thus the inhomogeneities do not impede high powers by the formation of quantum dots, quantum films or other boundary surfaces, e.g. by dispersion at boundary surfaces or boundary surface defects or at the quantum points.

The waveguide has a width of at least 200, 250, 300, 400, 500 or 600 nm. The waveguide is defined by a refractive index between the interior of the waveguide and the exterior of the waveguide. The waveguide is preferably formed such that only a transversal light mode sets in. However, two or three transversal modes can also be given, since thereby higher powers become possible, however, without losing a well-defined beam profile.

The waveguide may comprise GaₓIn₍₁₋ₓ₎AsᵤPᵥN_{w}Sb_{(1-u-v-w)} or it may consist thereof, wherein x, u, v, and w may adopt the values 0 to 1 and all intermediate values or values from all possible intermediate intervals and u+v+w is smaller than or equal 1.

The semiconductor laser may also be a quantum film laser, with a quantum film which comprises GaᵤIn₁₋ᵤAsₓN_{y}P_{1-x-y} or consists thereof, wherein u, x and y may adopt the values 0 to 1 or all intermediate values or values from all possible intermediate intervals as long as x+y≦1. With such materials are the desired output powers in the predetermined wavelength range possible. This is carried out by combining a relatively large amount of laser elements, so that the required overall output power of the laser appears to be reachable.

Solid-state disk layers are also advantageously possible for the generation of high performance lasers in the required wavelength range. In these layers a crystal has the shape of a disk and has a mirror glass on one side. The laser emerges on the opposite side. Such disks may be cooled very well so that high powers become possible.

In any case are cylindrical lenses and/or lens arrays and/or mirrors for coupling in the light of several laser elements advantageous.

The device comprises a coupling to which optical fibers can detachably be connected. During surgery, the optical fibers are easily soiled so that they should be exchangeable.

The optical fiber advantageously comprises a handle member, since this simplifies manipulation of the optical fiber. An optical system may also be provided in the handle member by means of which the light emerging from the optical fiber is focused onto a focus. However, this is not mandatory, since an irradiation portion of a diameter of several millimeters can also be achieved without an optical system.

The optical system is preferably also exchangeable. On the one hand, the optical system is also slightly soiled, on the other hand different working distances and different focal sizes can be achieved by different optical systems.

The device further comprises an air cooling for cooling the laser or the device. A water cooling is not provided, since the air cooling should be sufficient for a semiconductor laser. However, water cooling is also not excluded.

The device may further comprise a temperature control means by means of which the temperature of the semiconductor laser can be adjusted. This may for instance comprise a Peltier element. This on the one hand serves for discharging the waste heat. On the other hand, the wavelength of the emitted light can also be adjusted by the temperature. The Peltier element can be cooled by water and/or air. A water cooling without a Peltier element is also possible. The water cooling may also be quite small.

Preferred embodiments of the invention shall be explained by means of the enclosed Figures.

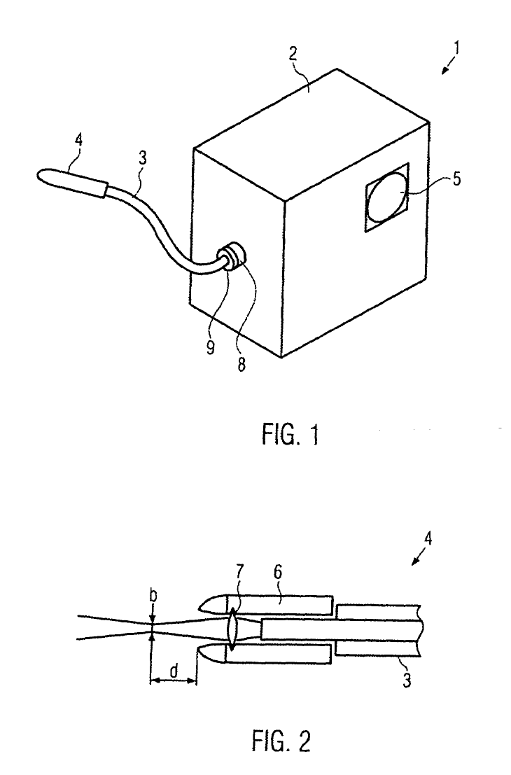

FIG. 1 shows a device for laser surgery;

FIG. 2 shows a schematic sectional view of the distal end of the optical fiber;

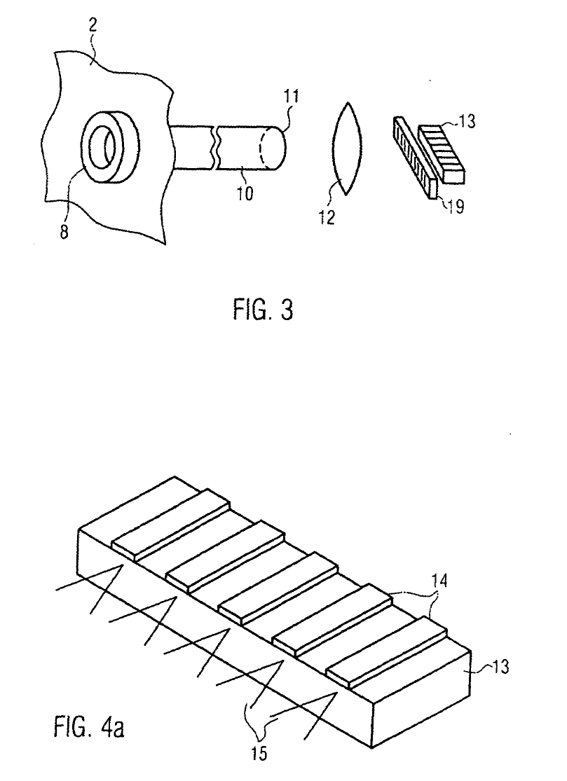

FIG. 3 shows the arrangement of the semiconductor laser; a coupling optical system and a fiber end in a schematic three-dimensional view;

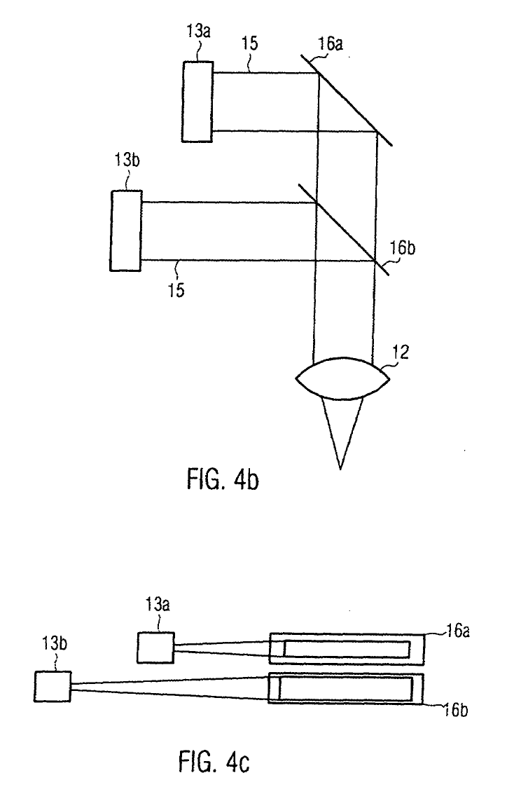

FIG. 4 shows schematic three-dimensional views of a laser arrangement;

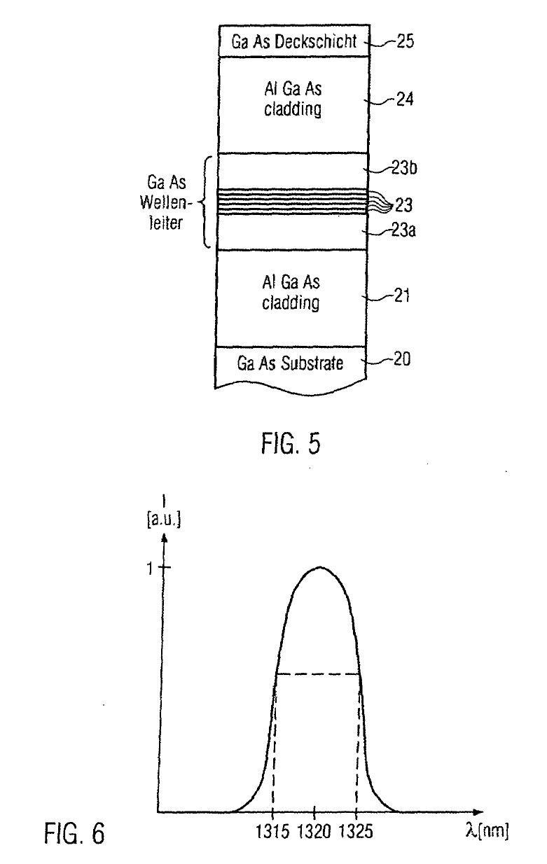

FIG. 5 shows a schematic view of the structure of a semiconductor quantum dot laser;

FIG. 6 shows an exemplary spectrum of the laser.

FIG. 1 shows a device for laser surgery. The device comprises a housing 2 with a coupling 8 into which an optical fiber 3 with a plug 9 can be coupled in. The optical fiber 3 has an end with a handle portion 4. The housing 2 also has a cooler 5 for the air cooling.

The end of the optical fiber 3 with the handle 4 is schematically shown in FIG. 2 in a sectional view. The optical fiber 3 ends in a detachable optical system holder 6 in which an optical system 7, symbolically shown by a single lens 7, is arranged. The light emerging from the optical fiber 3 is bundled by this optical system so that in a working distance d a focus with a full width at half maximum b is formed. The optical fiber 3 has a light-conducting fiber core as well as a coating. The preferred working distance d is some centimeters. Especially preferred is a working distance of 1 to 5 cm, such as 1.5 cm (±0.5 cm) or 2.5 cm (±1.0 cm) or 3.5 cm (±1.0 cm) or 4.5 cm (±0.5 cm). The full width at half maximum b in the focal range is some millimeters. The beam diameter in the focus may for instance be 0.5 mm.

FIG. 3 shows part of the housing 2 with the coupling 8. Here a fiber end 11 of an internal optical fiber 10 is schematically shown into which the light of the laser 13 is coupled. The laser 13 is designed here as a laser bar. Between the laser bar 13 and the end 11 of the optical fiber 10 a coupling optical system 12 is provided, by means of which the light divergently emerging from the bar 13 is bundled towards the optical fiber end 11. The coupling optical system may comprise a cylindrical lens or a cylindrical mirror. A cylinder lens array 19 is for instance preferred, wherein one cylinder lens element of the cylinder lens array is preferably associated to each laser element.

The bar 13 is schematically shown in FIG. 4a. Various semiconductor laser elements 14 are arranged in the bar, wherein a light laser beam 15, which may be very divergent, emerges from each element 14.

In the bar 13 in FIG. 4a laser elements 14 are arranged in juxtaposition. Such a laser bar may comprise up to 10, 15, 20, 25, 30, 35, 40, 45 or 50 laser elements 14.

FIGS. 4b and 4c show arrangements in top plan view and in front view, by means of which the light from different laser bars can be bundled. Two bars 13 a and 13 b are arranged in a manner offset in height. They emit the light towards one mirror 16a, 16b each, which are also arranged in a manner offset in height. An additional cylinder lens array may be arranged between the bars 13a, 13b and the mirrors 16a and 16b to obtain a (at least approximately) collimated beam 15. The beams reflected by the mirrors 16a, 16b extend in parallel and on top of one another so that they can be focused by one single coupling optical system 12. The optical path between the coupling optical system 12 and the laser bars 13a, 13b is advantageously approximately equally long.

A schematic view of the layer structure of the semiconductor fiber is shown in FIG. 5. A cladding layer 21 is grown onto the substrate 20. On this layer the optical waveguide is formed. The optical waveguide has a refractive index that is higher than the one of the cladding layer. Thus, the optical waveguide guides the light modes in the semiconductor material. Various layers 23 are arranged within the optical waveguide, which may be quantum films. In these quantum films 23 quantum dots can also be arranged which define the laser wavelength of the laser.

It is also possible to only provide quantum dots without quantum films.

The quantum dots may also have the shape of quantum dashes. These are quantum dots, that have an oblong shape, such as a long drawn hexagon or the like.

A further cladding layer 24 is applied onto the optical waveguide and additionally, a cover layer 25 is also arranged on the optical waveguide. The substrate 20 and the cladding layer 21 preferably have an identical doping. The cladding layer 24 and the cover layer 25 have opposite doping. The optical waveguide has for instance a width of 500 nm and can therefore be designated as LOC (Large Optical Cavity). The cladding layers may for instance comprise AlₓGa₁₋ₓAs or they may consist thereof. The aluminum content and the thickness of these layers may be adapted suitably. The thickness may for instance be 1.6 mm.

Generally, δ-doped layers may be provided in the active region to achieve high laser outputs.

The optical waveguide may for instance be or comprises GaAs. Aluminum, indium, phosphorous or the like can be added thereto. Its band gap is smaller than the one of the cladding layer 21, 24. The material of the optical waveguide 23 a, 23 b is preferably undoped.

If the quantum dots are for instance formed of InGaAs, they can also be formed in InGaAs quantum films, if the indium content in the dots is substantially higher.

The quantum films 23 may also be formed without quantum dots. For instance, GaInAsN may be provided as film material. These material layers may be arranged between GaAs and/or GaInAs. Both the quantum film materials as well as the barriers in between may be adapted with antimony (sb) or phosphorous. Thereby the more specifically desired wavelength can be adjusted.

Instead of GaInAsN GaInAsP can also be used as quantum film or quantum dot material.

FIG. 6 shows a typical spectrum of the laser light output by the laser device. In FIG. 6 the intensity is shown in arbitrary units over the wavelength in nanometers. The spectrum shown there has a full width at half maximum of 10 nanometers and a central wavelength of 1320 nanometers.

1. A device for laser surgery, comprising:
a laser and
means for coupling laser light of the laser into an optical fiber characterized in that
the laser is a solid-state laser, which is capable of irradiating laser light, which at maximum intensity has a wavelength in the range of 1100 to 1400 nm,
wherein the laser has at least an output power of 25 W.

2. A device as claimed in claim 1, characterized in that the wavelength range of the maximum intensity is above 1150, 1175, 1200, 1275, 1300, 1308 or 1310 nm.

3. A device as claimed in claim 1, characterized in that the wavelength range of maximum intensity is below 1370, 1350, 1330, 1325, 1300, 1275, 1250, 1225 or 1200 nm.

4. A device as claimed in claim 1, characterized in that the spectral full width at half maximum of the laser light is at least 2, 3, 4, 5, 7, 10, 12 or 15 nm.

5. A device as claimed in claim 1, characterized in that the spectral full width at half maximum of the laser light is at most 2, 3, 4, 5, 7, 10, 12, 15, 20, 25, 30, 40 nm.

6. A device as claimed in claim 1, characterized in that the output power of the laser is at least 30, 35, 40, 45, 50, 60, 70, 80, 90 or 100 W.

7. A device as claimed in claim 1, characterized in that the laser is a continuous-wave laser or pulsed laser.

8. A device as claimed in claim 1, characterized in that the laser comprises a plurality of laser elements, such as at least or exactly 2, 5, 10, 15, 19, 20, 25, 30, 35, 38, 40, 50, 60, 70, 80, 90, 100, 120 or 150 laser elements.

9. A device as claimed in claim 8, characterized in that means for coupling laser light of the individual laser elements into a common optical fiber are provided.

10. A device as claimed in claim 1, characterized in that the solid-state laser is or comprises a fiber laser.

11. A device as claimed in claim 1, characterized in that the solid-state laser is or comprises a semiconductor laser.

12. A device as claimed in claim 11, characterized in that the laser or the laser elements are or comprise quantum dot lasers and/or quantum film lasers.

13. A device as claimed in claim 12, characterized in that the quantum dot lasers comprise GaAs and AlGaAs or InP and AlGaAsP.

14. A device as claimed in claim 12, characterized in that the quantum dot laser/s comprise/s quantum dots in a quantum film or outside of a quantum film or in an optical waveguide material without a quantum film.

15. A device as claimed in claim 12, characterized in that the quantum dots comprise GaₓIn₁₋ₓAs, GaₓIn₁₋ₓAs_{y}P_{1-y}, GaₓIn₁₋ₓAs_{y}N_{1-y} and/or GaₓIn₁₋ₓSb or consist thereof, wherein x and/or y may adopt the values from 0 to 1.

16. A device as claimed in claim 11, characterized in that the optical waveguide (22) of the semiconductor laser has a width of at least 200, 250, 300, 400, 500 or 600 nm.

17. A device as claimed in claim 11, characterized in that the optical waveguide (22) comprises GaₓIn₍₁₋ₓ₎AsᵤPᵥN_{w}Sb_{(1-u-v-w)} or consists thereof, wherein x, u, v, and w may adopt the values 0 to 1 and all intermediate values or values from all possible intermediate intervals and u+v+w is smaller than or equal to 1.

18. A device as claimed in claim 12, characterized in that the quantum film laser comprise a quantum film, which comprises GaᵤIn₁₋ᵤAsₓN_{y}P_{(1-x-y)} or consists thereof, wherein y may adopt the values 0 to 1 or all intermediate values or values from all possible intermediate intervals as long as x+y≦1.

19. A device as claimed in claim 11, characterized in that the solid-state laser is or comprises a disk laser.

20. A device as claimed in claim 1, characterized in that a coupling is provided for the detachable connection of an optical fiber (3).

21. A device as claimed in claim 1, characterized in that the device comprises a detachable optical fiber.

22. A device as claimed in claim 21, characterized in that the optical fiber comprises a handle member.

23. A device as claimed in claim 21, characterized in that an optical system is provided at the distal end of the optical fiber by means of which a light focus can be formed at a distance (d) of 1 to 6 cm, preferably 1 to 5 cm, even more preferred 1.5 to 3 cm, from the end of the optical fiber, wherein the optical system is preferably exchangeable.

24. A device as claimed in claim 23, characterized in that the optical system generates a focus with a full width at half maximum (b) of 0.2 to 0.7 nm, preferably 0.3 to 0.6 nm and even more preferred 0.4 to 0.5 nm.

25. A device as claimed in claim 1, characterized in that an air cooling device for cooling the laser with cooling air is provided.
